(19) United States
(12) Patent Application Publication
(10) Pub. No.: US 2009/0032018 A1
(43) Pub. Date: Feb. 5, 2009

(54) System Adapted to Provide a Flow of Gas to an Airway of a Patient

(76) Inventors: Jason P. Eaton, Hunker, PA (US); Luke Christopher Stonis, Columbus, OH (US); John B. Youger, Columbus, OH (US); Christopher David von Dohlen, St. Louis Park, MN (US); Jeffrey William Servaites, San Francisco, CA (US); Christopher H. Kujawski, Columbus, OH (US); Andrew Barrow, Grove City, OH (US)

Correspondence Address: MICHAEL W. HAAS;RESPIRONICS, INC., 1010 MURRY RIDGE LANE, MURRYSVILLE, PA, 15668, US

(21) Appl. No.: 12/182,191

(22) Filed: Jul. 30, 2008

Related U.S. Application Data
(60) Provisional application No. 60/953,807, filed on Aug. 3, 2007.

(51) Int. Cl. A61M 16/00; A61M 16/20

(52) U.S. Cl. 128/201.22; 128/205.24

(57) ABSTRACT

A system adapted to provide a flow of gas to an airway of a patient. The system comprises a first conduit, a second conduit, an interface appliance, and a retention assembly. The first conduit has a proximal end and a distal end. The second conduit has a proximal end and a distal end. The interface appliance has an inlet opening, an outlet opening, and one or more airway openings. The inlet opening is engaged with the proximal end of the first conduit such that the flow of gas is provided from the first conduit to the interface appliance at the engagement between the inlet opening and the proximal end of the first conduit. The one or more airway openings are adapted to deliver a portion of the flow of gas to the airway of the patient. The outlet opening is engaged with the proximal end of the second conduit such that a portion of the flow of gas is released from the interface appliance into the second conduit at the engagement between the outlet opening and the proximal end of the second conduit. The retention assembly retains the interface appliance in position with respect to the airway of the patient by engaging the first and second conduits such that if the interface appliance is in position with respect to the airway of the patient the first conduit and the second conduit run laterally across the patient's face on each side of the interface appliance underneath the eyes of the patient.

### CROSS-REFERENCE TO RELATED APPLICATIONS

This application claims priority under 35 U.S.C. § 119(e) from provisional U.S. patent application No. 60/953,807 filed Aug. 3, 2007, the entire contents of which are incorporated herein by reference.

### BACKGROUND OF THE INVENTION

1. Field of the Invention

The invention relates to systems adapted to provide breathing gas to infants, including gas delivery systems that carry the gas to and from an infant's airway and retention systems that hold a gas delivery system in place on an infant during operation.

2. Description of the Related Art

Conventional systems adapted to provide breathing gas to infants are not well supported on infants. As a result, when an infant moves during treatment relative to the breathing gas delivery system, forces are generated on an interface between the infant and the breathing gas delivery system that are not well distributed and supported. Thus, these forces may be uncomfortable to the infant (e.g., at or around the nose or mouth), cause leaks, or produce other unsatisfactory results. To counteract these problems, caregivers may attempt to over-tighten supports that hold the gas delivery system in place, which may be undesirable. Other drawbacks with conventional devices are also known.

### SUMMARY OF THE INVENTION

One aspect of the invention relates to a system adapted to provide a flow of gas to an airway of a patient. In one embodiment, the system comprises a first conduit, a second conduit, an interface appliance, and a retention assembly. The first conduit has a proximal end and a distal end. The second conduit has a proximal end and a distal end. The interface appliance has an inlet opening, an outlet opening, and one or more airway openings. The inlet opening is engaged with the proximal end of the first conduit such that the flow of gas is provided from the first conduit to the interface appliance at the engagement between the inlet opening and the proximal end of the first conduit. The one or more airway openings are adapted to deliver a portion of the flow of gas to the airway of the patient. The outlet opening is engaged with the proximal end of the second conduit such that a portion of the flow of gas is released from the interface appliance into the second conduit at the engagement between the outlet opening and the proximal end of the second conduit. The retention assembly retains the interface appliance in position with respect to the airway of the patient by engaging the first and second conduits such that if the interface appliance is in position with respect to the airway of the patient the first conduit and the second conduit run laterally across the patient's face on each side of the interface appliance underneath the eyes of the patient.

Another aspect of the invention relates to a system adapted to provide a flow of gas to an airway of a patient. In one embodiment, the system comprises a first conduit, a second conduit, an interface appliance, and a retention assembly. The first conduit has a proximal end and a distal end. The second conduit has a proximal end and a distal end. The interface appliance has a first opening, a second opening, and one or more airway openings. The first opening is engaged with the proximal end of the first conduit. The one or more airway openings are adapted to be in fluid communication with the airway of the patient. The second opening is engaged with the proximal end of the second conduit. The retention assembly retains the interface appliance in position with respect to the airway of the patient. The retention assembly comprises clips that engage the first and second conduits between their distal and proximal ends such that near the engagements between the clips and the first and second conduits (i) the portions of the first and second conduits between the engagements and the proximal ends of the first and second conduits are oriented in directions that are roughly lateral to the patient's head and (ii) the portions of the first and second conduits between the engagements and the distal ends of the first and second conduits are oriented in directions that are roughly longitudinal to the patient's head.

Another aspect of the invention relates to a system adapted to provide a flow of gas to an airway of a patient. In one embodiment, the system comprises a first conduit, a second conduit, an interface appliance, and a retention assembly. The first conduit has a proximal end and a distal end. The second conduit has a proximal end and a distal end. The interface appliance has a first opening, a second opening, and one or more airway openings. The first opening is engaged with the proximal end of the first conduit. The one or more airway openings are adapted to be in fluid communication with the airway of the patient. The second opening is engaged with the proximal end of the second conduit. The retention assembly retains the interface appliance in position with respect to the airway of the patient. The retention assembly comprises clips that removably engage the first and second conduits between their distal and proximal ends.

Another aspect of the invention relates to a system adapted to provide a flow of gas to an airway of a patient. In one embodiment, the system comprises a first conduit, a second conduit, an interface appliance, and a retention assembly. The first conduit has a proximal end and a distal end. The second conduit has a proximal end and a distal end. The interface appliance has a first conduit opening, a conduit opening, and one or more airway openings. The first conduit opening is engaged with the proximal end of the first conduit. The one or more airway openings are adapted to communicate with the airway of the patient. The outlet opening is engaged with the proximal end of the second conduit. The retention assembly retains the interface appliance in position with respect to the airway of the patient. The retention assembly engages the first and second conduits such that if the interface appliance is in position with respect to the airway of the patient, the first conduit and the second conduit run laterally across the patient's face on each side of the interface appliance underneath the eyes of the patient.

Another aspect of the invention relates to a system adapted to provide a flow of gas to an airway of an patient. In one embodiment, the system comprises a first conduit, a second conduit, an interface appliance, and a retention assembly. The first conduit has a proximal end and a distal end. The second conduit has a proximal end and a distal end. The interface appliance has a first opening, a second opening, and one or more airway openings. The first opening is engaged with the proximal end of the first conduit. The one or more airway openings are adapted to be in fluid communication with the airway of the patient. The second opening is engaged with the proximal end of the second conduit. The retention assembly retains the interface appliance in position with respect to the airway of the patient. The retention assembly comprises a headgear that is adapted to be carried on the head of the patient and to retain the first and second conduit in position with respect to the head of the patient. The retention assembly is configured such that the position of the first and second conduits with respect to the head of the patient is adjustable by adjusting the position of the first and second conduits with respect to the headgear.

Another aspect of the invention relates to a flow coupling that couples a flow of gas from a gas source to one or more conduits that deliver at least a portion of the flow of gas to an airway of a patient. In one embodiment, the flow coupling comprises a flow inlet, an outlet port, a first chamber, a release valve, and a swivel. The flow inlet accepts the flow of gas into the flow coupling. The outlet port outlets at least a portion of the flow of gas to a first conduit. The first chamber formed within the flow coupling communicates the flow inlet with the flow outlet. The release valve releases gas from the first chamber to atmosphere, wherein the release valve is configured to release gas from the first chamber to atmosphere ensure that the pressure of the flow of gas delivered to the patient does not exceed a predetermined threshold. The swivel enables the flow inlet to rotate longitudinally independent from the outlet port.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a perspective view of a system adapted to provide a flow of gas to an airway of an infant, in accordance with one or more embodiments of the invention.

FIG. 2 is a perspective view of a gas delivery system, in accordance with one or more embodiments of the invention.

FIG. 3 is a cross-sectional view of an interface appliance, according to one or more embodiments of the invention.

FIG. 4 is a perspective view of a conduit couple, according to one or more embodiments of the invention.

FIG. 5 is a perspective view of a headpiece of a retention system adapted to retain a gas delivery system in place on an infant, in accordance with one or more embodiments of the invention.

FIG. 6A is a perspective view of a clip adapted to secure a conduit, in accordance with one or more embodiments of the invention.

FIG. 6B is a perspective view of a clip securing a conduit according to one or more embodiments of the invention.

FIG. 7 is a perspective view of a system adapted to provide a flow of gas to an airway of an infant, in accordance with one or more embodiments of the invention.

FIG. 8 is a perspective view of a system adapted to provide a flow of gas to an airway of an infant, in accordance with one or more embodiments of the invention.

FIG. 9 is a perspective view of a system adapted to provide a flow of gas to an airway of an infant, in accordance with one or more embodiments of the invention.

FIG. 10 is a perspective view of a system adapted to provide a flow of gas to an airway of an infant, in accordance with one or more embodiments of the invention.

FIGS. 11A and 11B are perspective views of a system adapted to provide a flow of gas to an airway of an infant, in accordance with one or more embodiments of the invention.

FIGS. 12A and 12B are perspective views of a system adapted to provide a flow of gas to an airway of an infant, in accordance with one or more embodiments of the invention.

FIG. 13 is a perspective view of a system adapted to provide a flow of gas to an airway of an infant, in accordance with one or more embodiments of the invention.

FIG. 14 is a perspective view of a system adapted to provide a flow of gas to an airway of an infant, in accordance with one or more embodiments of the invention.

FIG. 15 is a perspective view of a system adapted to provide a flow of gas to an airway of an infant, in accordance with one or more embodiments of the invention.

FIGS. 16A-16C are perspective views of a system adapted to provide a flow of gas to an airway of an infant, in accordance with one or more embodiments of the invention.

FIG. 17 is a perspective view of a system adapted to provide a flow of gas to an airway of an infant, in accordance with one or more embodiments of the invention.

FIG. 18 is a perspective view of a system adapted to provide a flow of gas to an airway of an infant, in accordance with one or more embodiments of the invention.

FIGS. 19A and 19B are perspective views of a system adapted to provide a flow of gas to an airway of an infant, in accordance with one or more embodiments of the invention.

### DETAILED DESCRIPTION OF THE EXEMPLARY EMBODIMENTS

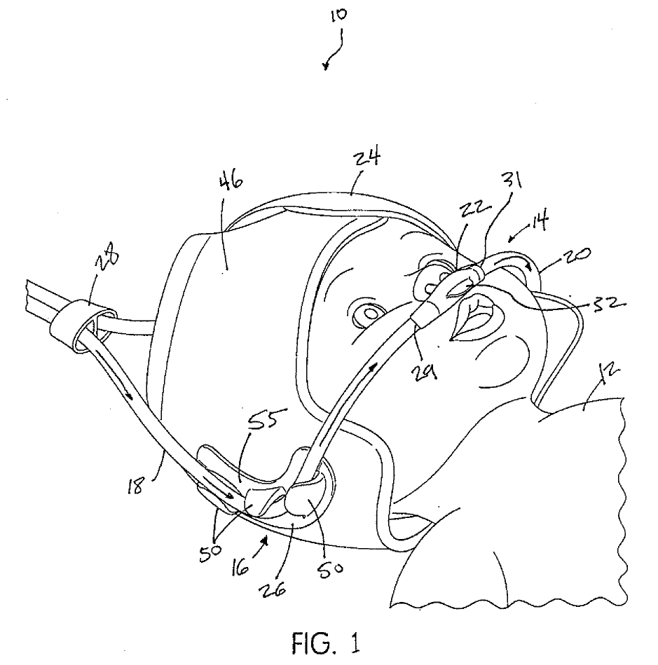

Turning to FIG. 1, a system 10 adapted to provide a flow of gas to an airway of an infant 12 is illustrated according to one or more embodiments of the invention. System 10 includes a gas delivery assembly 14 and a retention assembly 16. Gas delivery assembly 14 guides the flow of gas to and away from the airway of infant 12. Retention assembly 16 holds one or more components of gas delivery assembly 14 in place with respect to the airway of infant 12. In some embodiments, gas delivery assembly 14 comprises a first conduit 18, a second conduit 20, and an interface appliance 22. In some embodiments, retention assembly 16 comprises a head mount, or a headpiece 24, and one or more clips 26.

Conduits 18, 20 and interface appliance 22 provide for the delivery of the flow of gas from a gas source (not shown) to the airway of infant 12. The gas source may include, just for example, a PAP device, a ventilator, a pressurized gas storage device (e.g., a tank), wall gas, and/or other gas sources. The flow of gas may be provided to the airway of infant 12 for therapeutic purposes (e.g., to facilitate breathing, etc.). If desired, the flow of gas may be provided to the airway of infant 12 with a predetermined pressure, flow, composition, and/or other predetermined characteristics or properties.

In one embodiment, first conduit 18 communicates with the gas source and interface appliance 22 to provide the flow of gas from the gas source to interface appliance 22. Interface appliance 22 is adapted to provide at least a portion of the flow of gas to the airway of infant 12. Interface appliance 22 may comprise, for example, an endotracheal tube, a nasal cannula, a tracheotomy tube, a mask, or other patient interface appliances. Second conduit 20 communicates with interface appliance 22 to receive at least a portion of the flow of gas that is not delivered to the airway of infant 12. Second conduit 20 exhausts the received gas away from the airway of infant 12. It should be appreciated that in addition to the portion of the flow of gas that is not delivered to the airway of infant 12, second conduit 20 receives some or all of the gas that is exhaled by infant through the infant's airway. Second conduit 20 exhausts this gas away from the airway.

Exhausting the received gas and/or a portion of the flow of gas in a position that is relatively remote from the infant may provide one or more enhancements. For example, the exhaust may prove to be a source of auditory noise. Thus, by placing the exhaust relatively remotely from the infant (e.g., via second conduit 20) the noise pollution experienced by the infant due to system 10 may be reduced. As another example, within system 10 condensation may form during operation. At the locations) where gas is exhausted from system 10, condensation may also be exhausted. Accordingly, by providing the exhaust away from the infant, condensation that has formed within system 10 may also be kept away from the infant. In one embodiment, the exhaust provided at the distal end of second conduit 18 may be supplemented by an exhalation feature or a blow off valve located at or near interface appliance 22.

In one embodiment, headpiece 24 is secured to the head of infant 12 to mount system 10 to infant 12. Clips 26 are attached to headpiece 24, and are removably engaged with conduits 18, 20 to secure conduits 18, 20 in place with respect to infant 12. In one embodiment, conduits 18, 20 are formed from flexible plastic tubing.

In some embodiments, clips 26 are removably attached to headpiece 24 (e.g., by adhesive, static attraction, hook-and-loop fasteners, etc.) to enable their position to be adjusted, thereby enabling adjustment of the orientation and positioning of conduits 18, 20 with respect to infant 12. In other embodiments the attachment of clips 26 to headpiece 24 is not adjustable and can be attached by a mechanical interlock, for example. In still other embodiments, clips 26 are not removable from headpiece 24.

Retention assembly 16 may be designed such that if gas delivery assembly 14 and retention assembly 16 are in place (e.g., with interface appliance 22 engaged with the airway of infant 12 as shown in FIG. 1), infant 12 experiences an enhanced comfort over conventional systems. For example, clips 26 hold conduits 18, 20 such that they are directed from clips 26 to interface appliance 22 laterally with respect to the head of infant 12, underneath the eyes of infant 12. This may provide enhanced comfort to infant 12 over systems that provide conduits 18, 20 in a longitudinal direction between the eyes of infant 12 (e.g., less of a view obstruction, a reduced bulk, etc.). The lateral orientation of conduits 18, 20 also reduce an amount of upward force experienced by infant 12 (e.g., at the nose) that may be caused by overtightening of conduits 18, 20. As another example, clips 26 hold conduits 18, 20 such that conduits 18, 20 run from clips 26 toward ends opposite from the connections with interface appliance 22 in directions that are roughly longitudinal with respect to the head of infant 12. This enables conduits 18, 20 to meet at a region above the head of infant 12. Thus, infant 12 may lie down without lying on conduits 18, 20. Further, by providing conduits 18, 20 on opposite sides and above the head of infant 12, retention assembly 16 may enable infant 12 to actuate her head (e.g., by rolling, by twisting, etc.) with a reduced amount of interference from conduits 18, 20. The clip 26 also retains the conduits 18, 20 spaced from or suspended above the surface on which the infant rests her head. In one embodiment the clips 26 may be retained forwardly of the ears of infant 12 (i.e., closer to the cheeks of infant 12).

In some embodiments, retention assembly 16 may include a conduit link 28. Conduit link 28 holds conduits 18 and 20 together in close proximity to one another. This may reduce tangles and/or impediments to infant's ability to manipulate their head. Conduit link 28 is slidable along conduits 18 and 20 to enable adjustability.

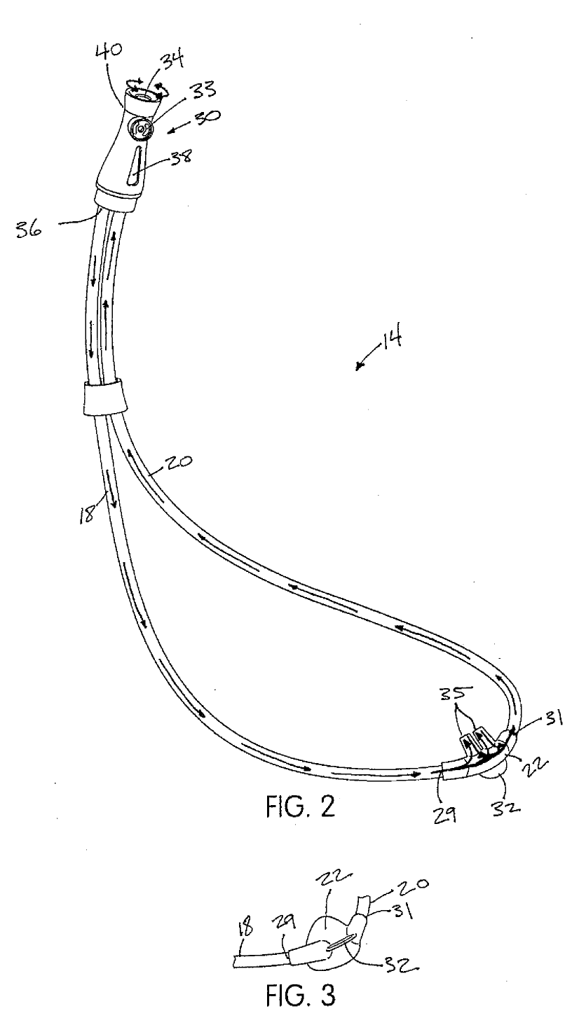

Referring to FIG. 2, an illustration of one embodiment of gas delivery assembly 14 is shown. As shown, gas delivery assembly 14 includes a conduit couple 30 that engages the ends of conduits 18, 20 (e.g., the distal ends) opposite from the ends of conduits 18, 20 that engage interface appliance 22 (e.g., the proximal ends). As was discussed above, first conduit 18 is arranged to carry the flow of gas from conduit couple 30 to interface appliance 22.

Interface appliance 22 is formed such that inlet opening 29 communicates with one or more interface openings 35 that deliver gas to an infant, and an outlet opening 31 formed by interface appliance 22 that is coupled with second conduit 20. In one embodiment (e.g., as shown in FIG. 2), the interface assembly 22 takes the form of a nasal cannula. As is illustrated in FIG. 2, interface appliance 22 receives the flow of gas from first conduit 18 at an inlet opening 29. Interface assembly 22 provides at least a portion of the flow of gas to the airway of the infant, via interface openings 35, and at least a portion of the flow of gas that is not delivered to the infant is delivered to second conduit 20 via outlet opening 31. As the infant breathes, the infant will exhale gas back into interface assembly through interface openings 35. As is shown in FIG. 2, the exhaled gas is exhausted from interface appliance 22 (along with a portion of the flow of gas) into second conduit 20 at outlet opening 31. Thus, first conduit 18, interface appliance 22, and second conduit 20 form a “blow-by system” that provides a supply of “fresh,” breathable air to the infant, while quickly exhausting exhaled gases.

In another embodiment, system 10 can provide a sidestream function. Specifically, rather than delivering oxygen, one or more of interface openings 35 may only receive exhaled carbon dioxide from the infant for sampling by a carbon dioxide monitor, as known in the art. In another embodiment, both conduits 18, 20 can provide oxygen delivery, or both can provide a carbon dioxide sampling function.

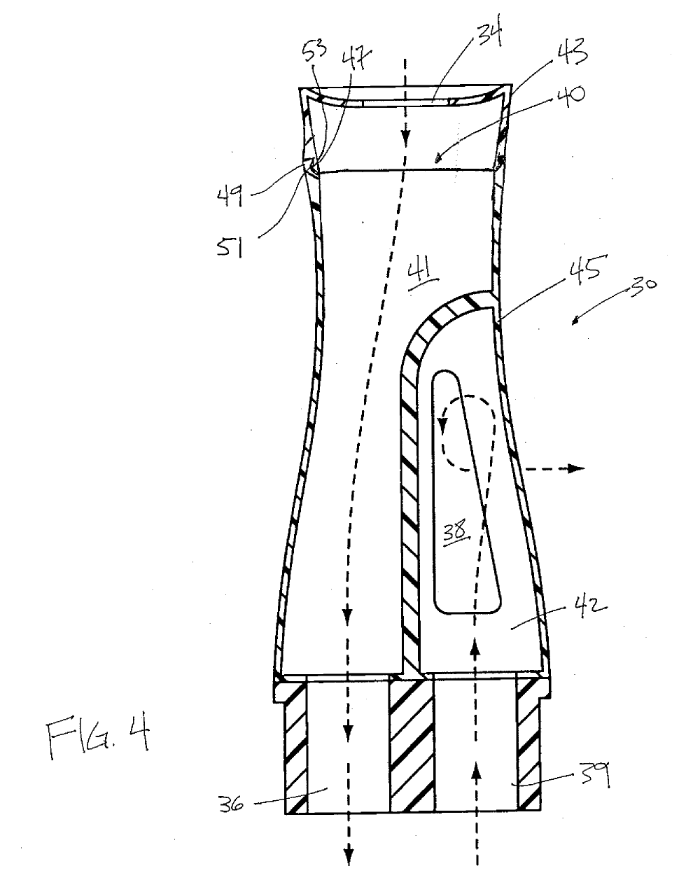

In the embodiment illustrated in FIG. 2, interface appliance 22 includes a nasal cannula with two separate ports, or openings, for each nostril. However, other interface assemblies with other opening configurations are contemplated. For example, FIG. 3 illustrates one possible alternative interface appliance 22 formed as a mask that fits over the nose of the infant. Similar to interface appliance 22 shown in FIGS. 1 and 2, interface appliance 22 illustrated in FIG. 3 is configured to receive the flow of gas from first conduit 18, provide at least a portion of the flow of gas to the airway of the infant, and exhaust gas via second conduit 20. It should be appreciated that other alternatives may be implemented.

Interface assemblies 22 shown in FIGS. 2 and 3 may optionally include a protrusion 32 that can be grasped by a user (e.g., a caregiver) to remove interface appliance 22 from engagement with the airway of the infant.

Referring back to FIG. 2, conduit couple 30 includes a valve 33. Valve 33 may enable gas being delivered to first conduit 18 from gas delivery assembly 14 to be released in a controllable manner. For example, valve 33 may be closed (or partially closed) during typical operation, and may be opened to provide relief from the delivery of gas for the infant (e.g., during respiratory events, etc.). Conduit couple 30 further includes a flow inlet 34, a flow outlet port 36, an exhaust 38, and a swivel 40. In one embodiment, valve 33 may be configured to release gas as a control mechanism to ensure that the pressure within first conduit 18 (e.g., the gas being delivered to the infant) does not exceed a predetermined threshold.

Swivel 40 enables flow inlet 34 to swivel, or rotate about a longitudinal axis, with respect to conduits 18, 20 (e.g., in the directions illustrated by arrows in FIG. 3). This provides additional comfort and/or convenience in the implementation of system 10. For example, through operation of swivel 40, if an infant that is receiving gas from gas delivery assembly 14 rolls her head, the subsequent twisting of conduits 18, 20 in response to the rotation of the infant's head is not transferred (or not fully transferred) to flow inlet 34. This may enhance the comfort of the infant and/or reduce maintenance (e.g., untwisting conduits) performed by a user supervising the treatment of the infant. Further, swivel 40 enables (i) the connection between first conduit 18 and the gas source to be maintained and (ii) the delivery of the flow of gas through conduit couple 30 to be continued during and/or after rotational motion of the head of the infant (e.g., due to rolling, etc.).

Turning to FIG. 4, various aspects of the operation of flow inlet 34, flow outlet port 36, an exhaust port 39, exhaust 38, and swivel 40 are illustrated by a cross-section of conduit couple 30. As is illustrated in FIG. 4, flow inlet 34 is formed to receive a flow of gas from a gas source. Flow outlet port 36 communicates with flow inlet 34 by virtue of a channel 41 formed within conduit couple 30. Flow outlet port 36 is configured to engage the distal end of first conduit 18 to deliver the flow of gas from channel 41 into first conduit 18 (as shown, for example, in FIG. 2). As has been discussed above, first conduit 18 then provides the flow of gas to interface appliance 22. Exhaust 38 is provided to exhaust gas from within a chamber 42 formed within conduit couple 30 that communicates with second conduit 20 such that gas is provided from second conduit 20 to chamber 42. The gas is provided from second conduit 20 to chamber 42 by exhaust port 39, which is adapted to engage the distal end of second conduit 20. The gas within chamber 42 is exhausted from gas delivery assembly 14 to atmosphere by exhaust 38. It should be appreciated that exhaust 38 may be provided at other locations along second conduit 20 other than conduit couple 30.

In some embodiments, swivel 40 is formed at an interface between an upper body portion 43 and a main body portion 45 of conduit couple 30. As can be seen in FIG. 4, the interface is formed between a rim 47 of upper body portion 43 and a rim 49 of main body portion 45. Specifically, rim 47 fits within an opening formed by rim 49. In one embodiment, main body portion 45 includes a groove 51 formed at rim 49, and upper body portion 43 includes a ridge 53 that protrudes from rim 47. When upper body portion 43 is coupled to main body portion 45, ridge 53 engages groove 51 to retain rim 47 within 49 while allowing upper body portion 43 to rotate with respect to main body portion 45.

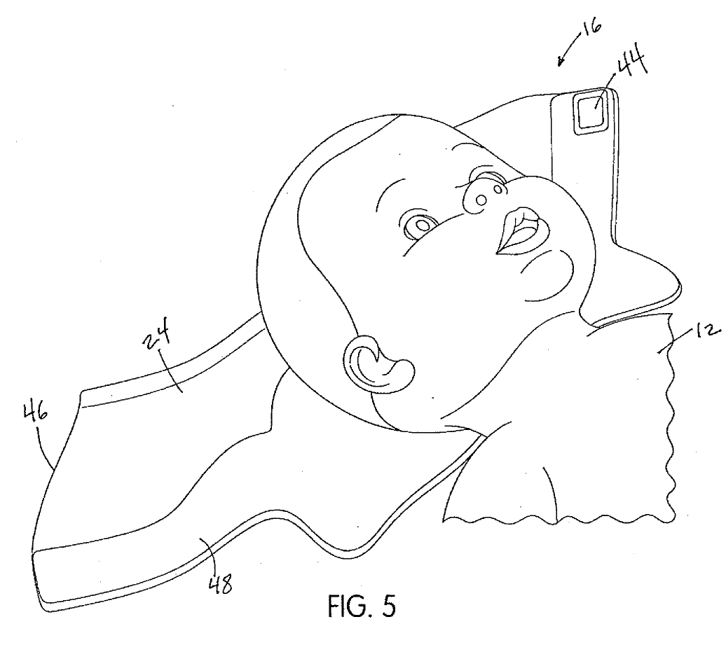

FIG. 5 illustrates various aspect of the operation of retention assembly 16, according to one or more embodiments of the invention. In the view shown in FIG. 5, headpiece 24 is prepared for application to the head of infant 12. In one embodiment, headpiece 24 is adapted to be applied to the head of infant 12 by laying infant 12 on top of headpiece 24 and then wrapping headpiece 24 around the head of infant 12. Headpiece 24 includes a fastener 44 that secures headpiece 24 once it has been wrapped around the head of infant 12. For example, in one embodiment, fastener 44 may form part of a hook-and-loop fastener arrangement as described below. However, other fastening arrangements can be used, such as adjustable straps, multiple snaps, etc.

In one embodiment, headpiece 24 includes a main body 46 and a lining 48. Main body 46 is formed from a soft, pliable material that provides for the comfort of infant 12. In some embodiments, main body 46 is formed from a cloth or other material that provides a “loop side” of a hook and loop fastener over selected portions of the outer surface provided by main body 46, up to and including the entirety of the outer surface. This may facilitate the removable attachment of fastener 44 (which forms the “hook side” of the hook-and-loop fastener) in securing headpiece 24 to the head of infant 12, the removable attachment of clips 26 to the outer surface of headpiece 24, and/or other removable attachments. Lining 48 is formed from a material that will enhance an amount of friction between the inner surface of headpiece 24 and the head of infant 12. This may reduce slippage of headpiece 24 on the head of infant 12. In some instances, the inclusion of lining 48 may enable additional padding to be provided within headpiece 24 between main body 46 and lining 48. In one embodiment, both main body 46 and lining 48 are formed to provide a limited amount of stretch so that residual compressive forces on the head of infant 12 due to overtightening by a supervising user may be kept within a reduced range. In some instances, main body 46 and/or lining 48 are formed to be breathable.

In one exemplary embodiment, main body 46 may be formed from a nylon UBL fabric. The nylon UBL fabric may combine elasticity, breathability, and a hook-receptive “loop” surface. In one embodiment, lining 48 is formed from a soft, breathable, slip-resistant urethane foam, which may be mechanically bonded to a fabric substrate (e.g., main body 46). In another embodiment, lining 48 may be formed from one or more other slip-resistant materials such as foam, silicone, and/or high coefficient-of-friction thermoplastic elastomer materials. In addition to the composition of lining 48, the size and or shape of lining 48 may be modified to optimize the non-slip performance of lining 48.

While FIG. 5 illustrates headpiece 24 as a being applied to infant 12 by laying headpiece 24 on a surface, positioning infant 12 with her head on headpiece 24, and then wrapping headpiece 24 around her head and fastening it in place with fastener 44, it should be appreciated that this is not intended to be limiting. Such a configuration may facilitate application of headpiece 24 to the head of infant 12 with a reduced amount of manipulation of infant 12. However, other configurations are contemplated. For instance, headpiece 24 may be formed as a hat that is pulled into place over the head of infant 12. Further, the size and shape of headpiece 24 shown are for illustrative purposes, and other configurations of headpiece 24 may be implemented (e.g., a smaller “headband” configuration, etc.).

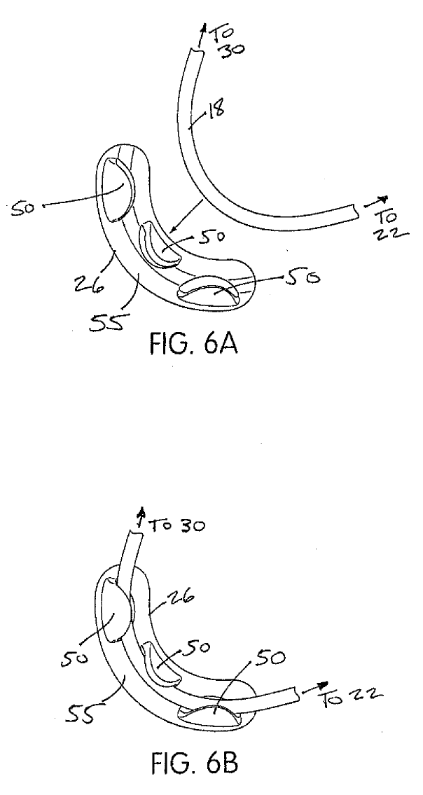

Turning to FIG. 6A, clip 26 is shown separate from conduit 18. As shown in FIG. 6A, clip 26 includes a main body 55 and a plurality of flanges 50. Flanges 50 are formed on main body 55 to protrude therefrom to engage conduit 18, thereby removably securing conduit 18 to clip 26, as is illustrated in FIG. 6B. Specifically, the body 55 and each respective flange 50 form a channel or gap therebetween sized to receive and grippingly engage an outer surface of conduit 18. Flanges 50 are formed with an arched shape to accommodate the shape of conduit 18 and increase the security of the engagement between flanges 50 and conduit 18.

In one embodiment, flanges 50 are disposed on clip 26 such that if conduit 18 is engaged by flanges 50, conduit 18 is flexed into a curved path defined by the placement and/or shape of flanges 50 on clip 26. For example, in some instances, the path formed by flanges 50 is arcuate. Since the flexure of conduit 18 is resilient, the elasticity of conduit 18 causes conduit 18 to attempt to flex back into a relatively straight configuration, thereby strengthening the engagement of conduit 18 by flanges 50.

The curvature of the path formed by flanges 50 causes the orientation of conduit 18 leaving clip 26 in one direction (e.g., toward the engagement with interface appliance 22 at its proximal end) to form a predetermined angle with the orientation of conduit 18 leaving clip 26 in a direction opposite from the first direction (e.g., toward the engagement with conduit couple 30 at its distal end). In some implementations, the orientations of the two adjacent sections of conduit 18 may be approximately perpendicular. This predetermined angle between two adjacent sections of conduit 18 may be in the range of between about 60° and 120°, and may facilitate some of the features discussed above. For instance, this enables the section of conduit 18 between clip 26 and interface appliance 22 to lie laterally along the head of infant 12 and underneath an eye of infant 12 (e.g., see FIG. 1), while the opposing section of conduit 18 lies along the head of infant 12 in a direction that is longitudinal to the head (e.g., see FIG. 1). As was mentioned previously, providing conduit 18 laterally underneath the eye of infant 12 to interface appliance 22 and curving the path of conduit 18 (e.g., by virtue if the path formed in clip 26) to leave retention assembly 16 from roughly the top of the head of infant 12 may enhance the comfort and/or usability of system 10.

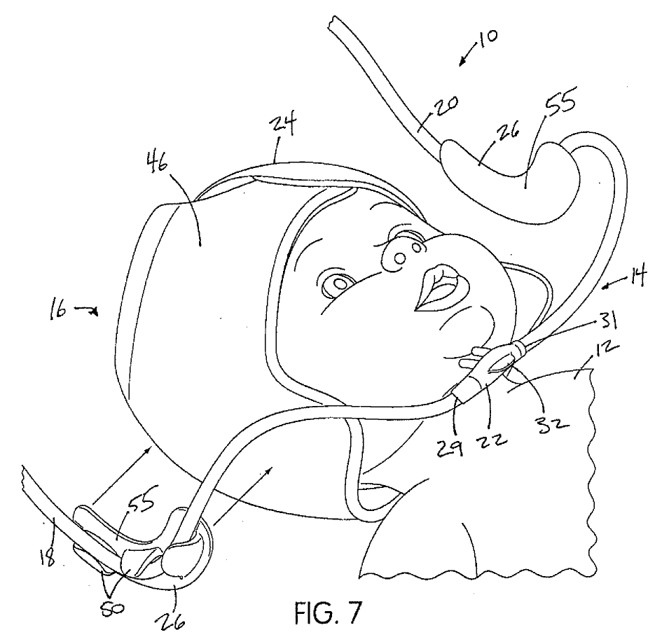

FIG. 7 illustrates a view of system 10 in which headpiece 24 has been secured to the head of infant 12, and conduits 18 and 20 have been engaged by clips 26. As is shown in FIG. 7, once headpiece 24 is in place on infant 12, clips 26 are attached to the outer surface of headpiece 24. In attaching clips 26 to headpiece 24, a supervising user may customize the “fit” of system 10 on infant 12. For example, the paths of conduits 18 and 20 may be set. Specifically, adjustment may be made for the relative size and/or shape of the head of infant 12, the particular facial geometry of infant 12, and/or other customizations may be made. The removable attachment of clips 26 to headpiece 24 enables this customization to be further refined during use. It also enables the same components from retention assembly 16 to be used for infants with a variety of different sizes and/or shapes of heads and a variety of facial geometries. In one embodiment, the removable attachment is enabled by a hook-side surface 52 provided on clips 26 that engages with the loop-side surface provided by main body 46 of headpiece 24. Other mechanisms for providing removable attachment of clips 26 to headpiece 24 are also contemplated (e.g., static attraction, adhesive, etc.). In addition the fastener portion 44 and cooperative fastener portion (material) 46 provides for additional adjustability.

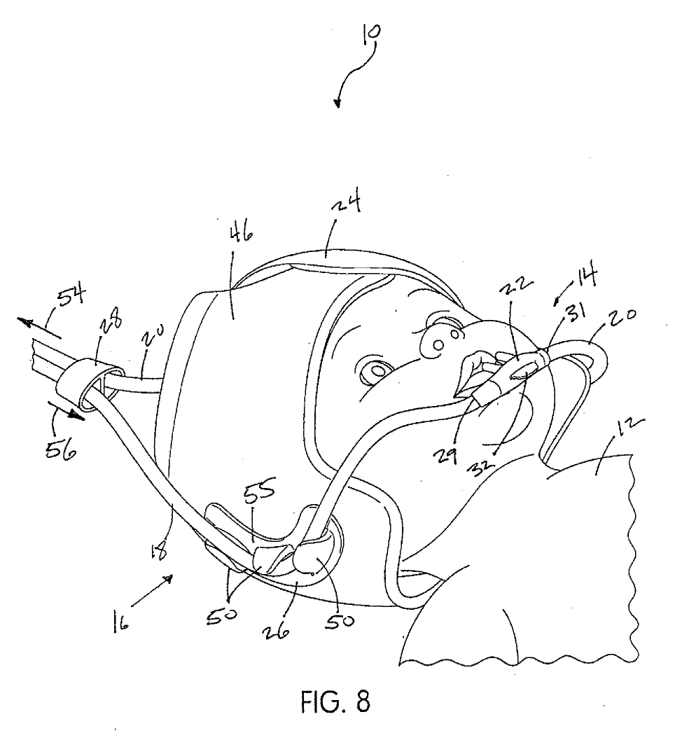

Referring now to FIG. 8, once clips 26 are in place on headpiece 24, gas delivery assembly 14 is adjusted to place interface appliance 22 in engagement with the airway of infant 12. For example, a tensile force is applied to conduits 18 and 20 (e.g., along arrow 54) that draws conduits 18 and 20 through clips 26 to take up slack in the sections of conduits 18 and 20 between clips 26 and interface appliance 22. This brings interface appliance 22 toward the airway of infant 12. In some instances, conduit link 28 is moved down conduits 18 and 20 (e.g., in the direction of arrow 56) to account for the drawing of conduits 18 and 20 through clips 26. Moving conduit link 28 down conduits 18 and 20 further secures gas delivery assembly 14 in place with respect to infant 12 and may reduce tangling of conduits 18 and 20 in response to motion by infant 12 and/or provide other efficiencies. Once these adjustments are made, gas delivery assembly 14 is in place with interface appliance 22 engaged with the airway of infant 12 and conduits 18, 20 secured by clips 26 in headpiece 24, as illustrated in FIG. 1.

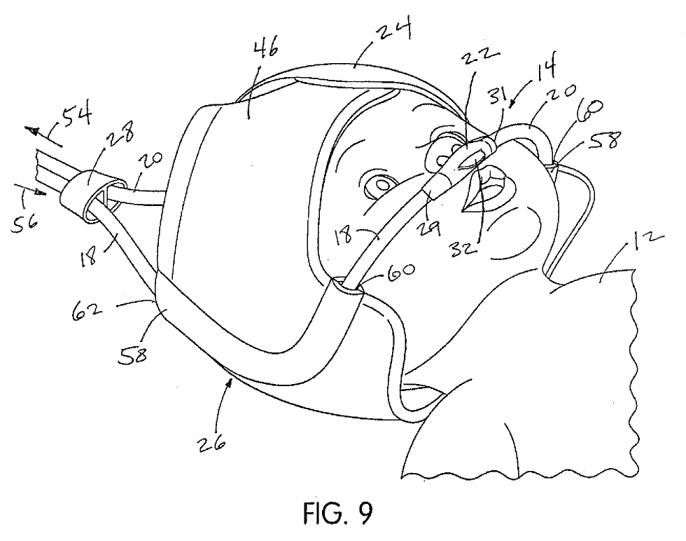

FIG. 9 illustrates a configuration of system 10, according to another embodiment of the invention, in which each of clips 26 include a channel 58 integrally formed in headpiece 24. Channel 58 may run from a proximal opening 60, located at or near the edge of headpiece 24 proximate to the face of infant 12, to a distal opening 62, located at or the edge of headpiece 24 adjacent to the top of the head of infant 12. The paths created for conduits 18 and 20 at distal openings 62 may be oriented a predetermined angle with respect to the path of conduits 18 and 20 at proximal openings 60. The predetermined angle may be determined to ensure that conduits 18 and 20 will be oriented to run across the face of infant 12 to the crown of the head of infant 12 (e.g., for the reasons discussed above). For example, the predetermined angle may be between about 60° and about 120°. In one embodiment, the predetermined angle may be approximately 90°. Channels 58 may be formed from a material that provides a relatively non-slip interface with first and second conduits 18 and 20 (which may be formed of, for example, vinyl tubing). This may prevent slippage of conduits 18 and 20 within channels 58 during use.

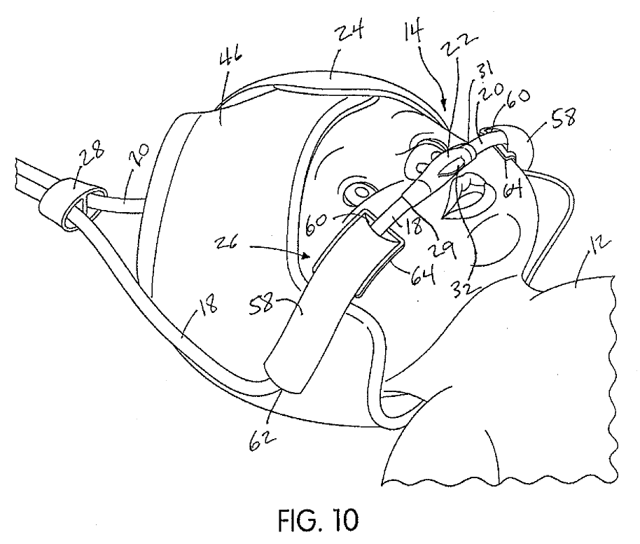

FIG. 10 illustrates system 10, according to one embodiment of the invention, that includes an alternate configuration of channels 58. In the configuration shown in FIG. 10, channels 58 provide a relatively straight path between proximal opening 60 and distal opening 62. To enable conduits 18 and 20 to run laterally across the face of infant 12 and up to the top of the head of infant 12, distal openings 62 are located on the side of the head of infant 12 (when infant 12 is wearing headpiece 24). In the embodiment shown, channels 58 extend past the edge of headpiece 24 onto the face of infant 12 such that proximal openings 60 are located on the cheeks of infant 12, below the eyes of infant 12. A pad 64 may be provided between channel 58 and infant 12 on the portion of channel 58 that extends from headpiece 24, in order to enhance the comfort of infant 12.

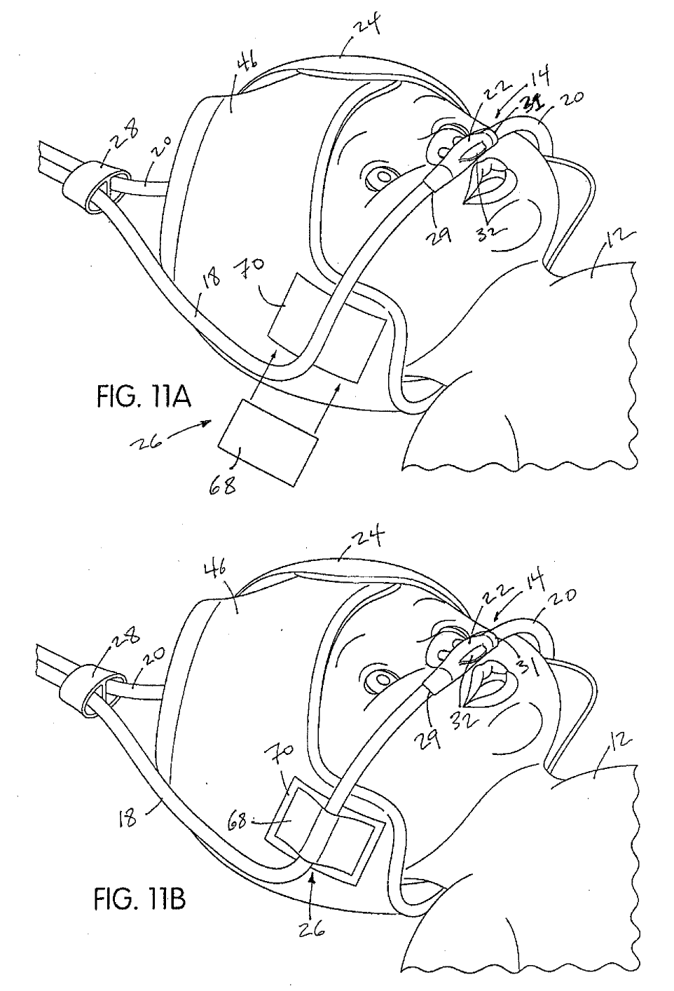

FIGS. 11A and 11B illustrate a configuration of system 10, according to one embodiment of the invention, in which each of clips 26 include a flexible strip 68 that is completely detachable from headpiece 24. Strip 68 may be removably attachable to headpiece 24 via hook-and-loop fasteners, adhesives, static attraction, and/or other mechanisms. In one embodiment, strip 68 provides an adhesive surface that can be removably adhered to complimentary surface 70 provided on headpiece 24. In using the configuration of system 10 illustrated in FIGS. 11A and 11B, conduits 18 and 20 are positioned appropriately with respect to infant 12, and then strips 68 are placed over conduits 18 and 20, thereby securing conduits 18 and 20 in place on headpiece 24.

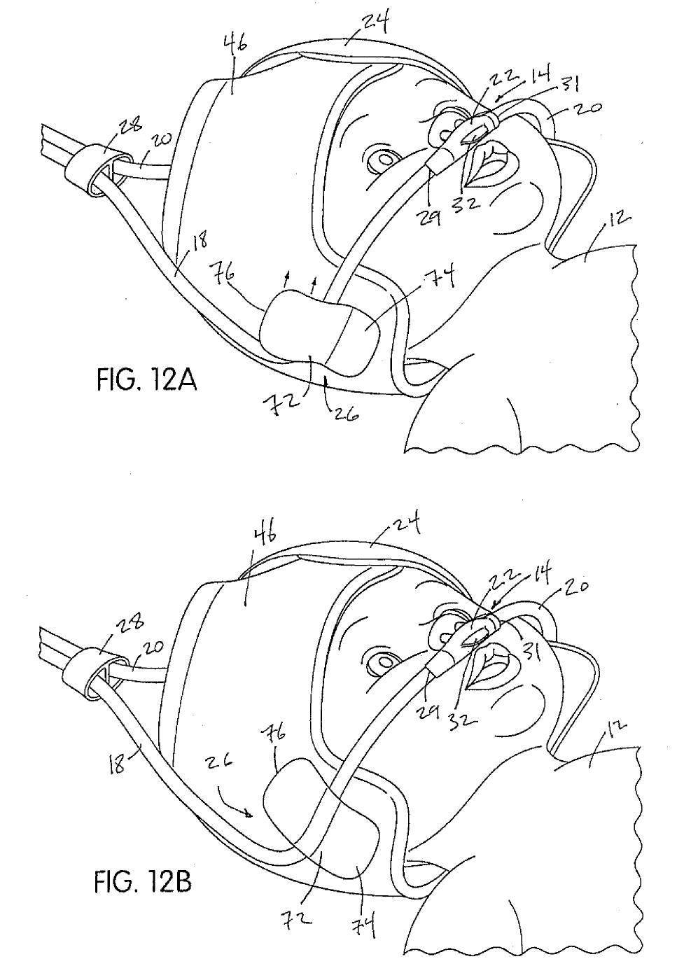

FIGS. 12A and 11B illustrate a configuration of system 10, according to one embodiment of the invention, in which each of clips 26 include a flexible flap 72 that is permanently affixed (or substantially so) to, or formed integrally with, headpiece 24 on a first side 74 and is removably attachable to headpiece 24 on a second side 76. Second side 76 of flap 72 may be removably attachable to headpiece 24 via hook-and-loop fasteners, adhesives, static attraction, and/or other mechanisms. In using the configuration of system 10 illustrated in FIGS. 12A and 12B, conduits 18 and 20 are positioned appropriately with respect to infant 12, and then flaps 72 are placed over conduits 18 and 20, thereby securing conduits 18 and 20 in place on headpiece 24.

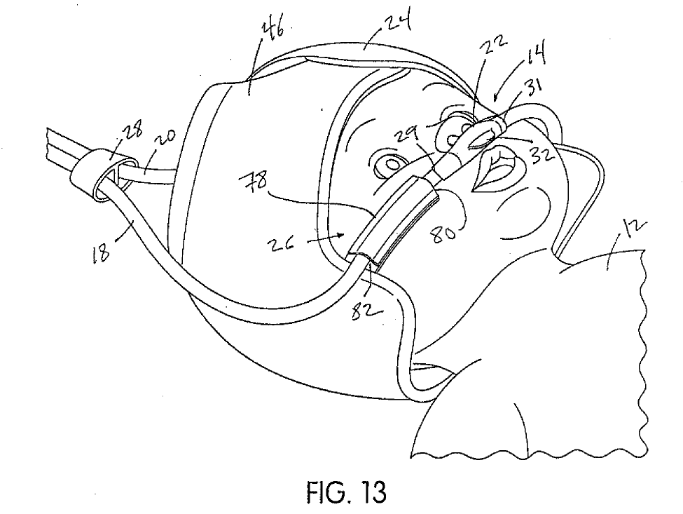

FIG. 13 illustrates a configuration of system 10, according to one embodiment of the invention. In the configuration illustrated in FIG. 13, on one side of the head of infant 12 clip 26 includes a channel 78 adapted to receive first conduit 18 therethrough. Channel 78 extends from headpiece 24, and is configured to lie laterally across the face of infant 12 at roughly the cheek. Channel 78 runs from a proximal opening 80 to a distal opening 82. In one embodiment, distal opening 82 may be located at or near the edge of headpiece 24 (as illustrated). In another embodiment, channel 78 may extend from proximal opening 80 onto the body of headpiece 24 (e.g., in a configuration similar to that shown in FIG. 10) such that distal opening 82 is formed on headpiece 24. On the side opposite channel 78, second conduit 20 is secured in place by clip 26 including a “quick release” mechanism that enables second conduit 18 to be quickly removed from headpiece 24. For example, in one embodiment, second conduit 20 is secured in place on headpiece by a clip similar to clip 26 shown in FIGS. 4 and 6-8, and described above, a removable strip similar to removable strip 68 shown in FIGS. 11A and 11B, and described above, a flap similar to flap 72 shown in FIGS. 12A and 12B, and described above, and/or other quick release mechanisms. The configuration illustrated in FIG. 13, in which one of conduits 18 and 20 is held in place by a channel 78 through which the given one of conduits 18 and 20 runs and the other of conduits 18 and 20 is held in place by a quick release mechanism, may fuse some advantages of embodiments in which conduits 18 and 20 are held in place on headpiece 24 in a fixed manner with some advantages of embodiments in which conduits 18 and 20 are held in place on headpiece 24 in a more easily removable manner. For example, this configuration may position conduits 18 and 20 with respect to infant with an enhanced degree of security (e.g., due to the relatively permanent positional support provided by channel 78) with an enhanced degree of customization, convenience (e.g., quick removal and/or release of conduits 18 and/or 20).

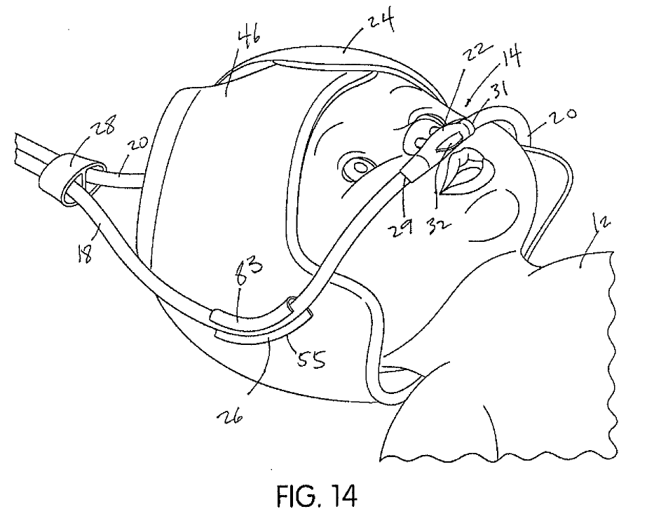

FIG. 14 illustrates a configuration of system 10, according to one embodiment of the invention, in which flanges 50 of clips 26 have been replaced by a single flange 83 that is configured to securely grip conduits 18 and 20. Flange 83 is shaped such that a cross-sectional view of clip 26 has the shape of a hook, wherein the arched portion of the hook corresponds to the circumference of conduits 18 and 20. As such, flange 83 is configured so that conduits 18 and 20 may be removably secured by flange 83 by inserting a given one of conduits 18 or 20 into the channel formed by the arched portion of the cross section of flange 83. Flange 83 may be arranged on clip 26 such that the opening provided by flange 83, into which one of conduits 18 or 20 may be inserted, faces generally away from the face of infant 12.

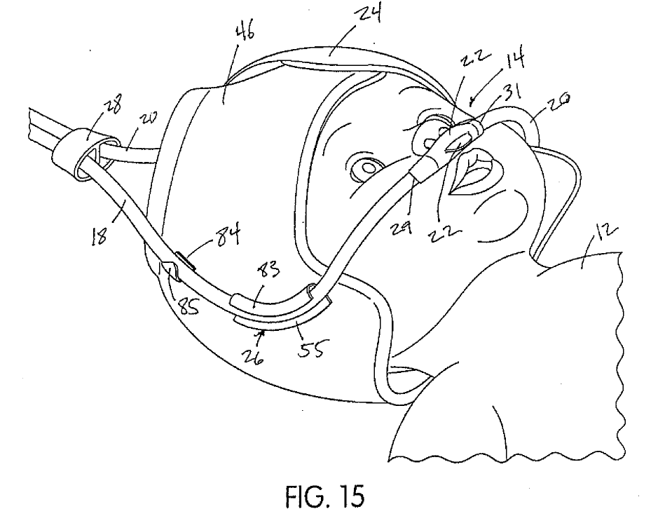

Turning to FIG. 15, in one embodiment, the engagement between clip 26 and conduit 18 or 20 may be further enhanced by a secondary clip 84 provided to retain conduit 18 or 20 within the channel provided by flange 83. Secondary clip 84 may include a flange 85 designed to engage conduit 18 or 20 to removably secure conduit 18 or 20 within a channel formed by flange 85 and the body of secondary clip 84.

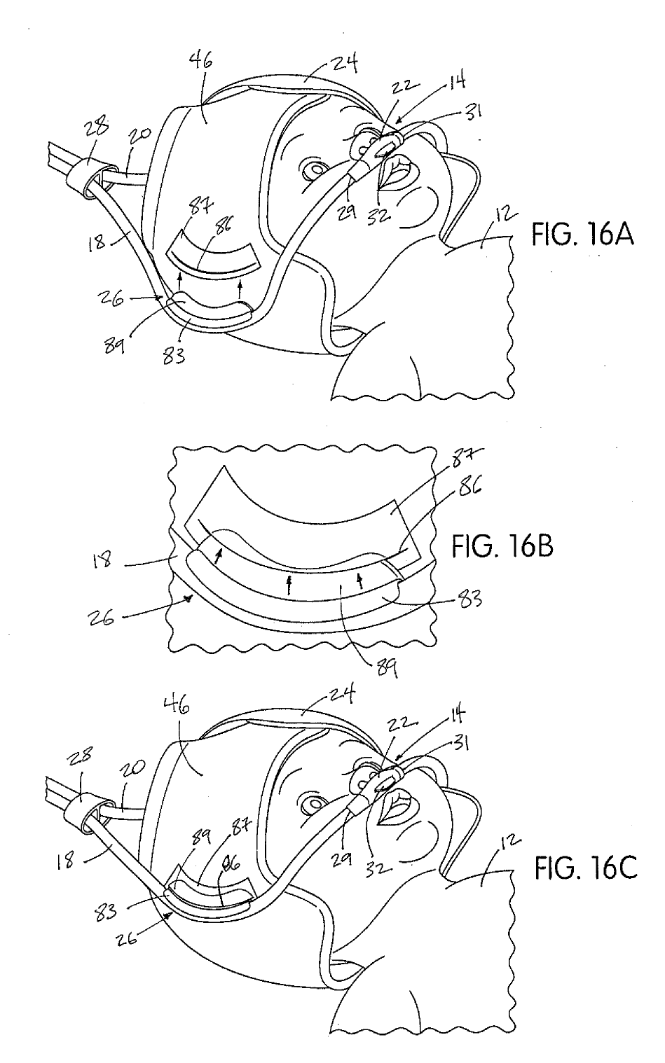

As has been mentioned above, in some embodiments clip 26 may be removable from headpiece 24. For example, FIGS. 16A-16C illustrate an implementation of this embodiment. In the implementation illustrated in FIGS. 16A-16C, headpiece 24 is formed with a slot 86 therein. Slot 86 may be provided on headpiece 24 by including slot 86 in an overlay 87 that is attached to the outer surface of headpiece 24. Clip 26 includes a tab 86 that protrudes therefrom and is adapted to slide into slot 86 (e.g., as is demonstrated in 16B) to removably secure clip 26 to headpiece 24 (e.g., FIG. 16C shows clip 26 secured to headpiece 24 with tab 86 securely nested within slot 86). This may provide some of the enhancements associated with a “quick release” mechanism (e.g., convenience, etc.), while increasing the ease with which clip 26 may be provided at a proper position on headpiece 24 (e.g., the position at slot 86) when clip 26 is reattached to headpiece 24.

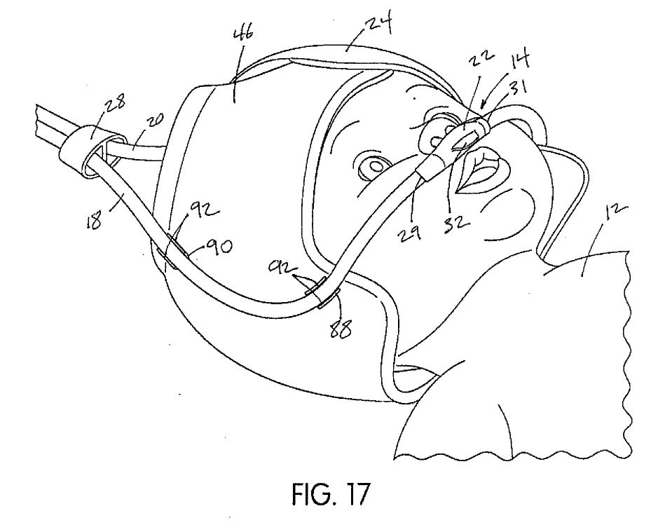

FIG. 17 illustrates system 10, according to one embodiment of the invention, in which conduits 18 and 20 are secured to headpiece 24 by a proximal clip 88 and a distal clip 90. Clips 88 and 90 each configured with a pair of prongs 92 adapted to grip a portion of conduit 18 or 20 placed therebetween. Proximal clip 88 is located on headpiece 24 proximate to the face of infant 12 (e.g., at or near the edge of headpiece 24 bordering the face of infant 12), and is oriented to route conduit 18 or 20 laterally across the face of infant 12. Distal clip 90 is located on headpiece 24 away from the face of infant 12, toward the top of the head of infant 12 and is oriented to route conduit 18 or 20 toward the top of the head of infant 12 at a predetermined angle from the route of conduit 18 or 20 across the face of infant 12. The predetermined angle may be between about 60° and about 120°. In one embodiment, the predetermined angle may be approximately 90°. Clips 88 and 90 may be permanently attached to headpiece 24, or clips 88 and 90 may be removably attached to headpiece 24.

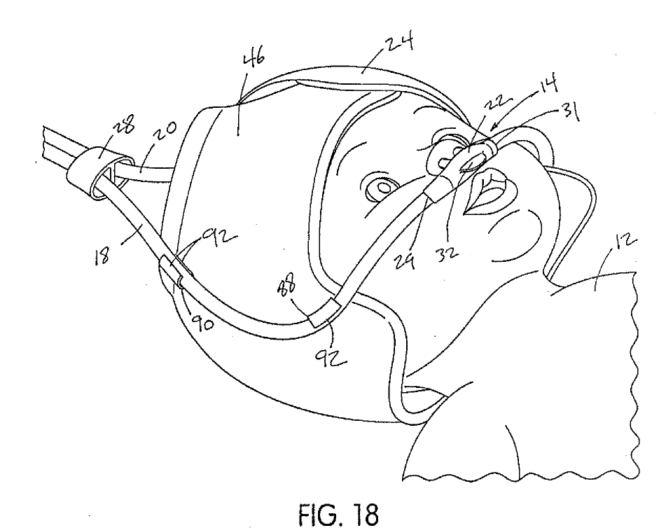

Turning to FIG. 18, in one embodiment, clips 88 and 90 are attached (and/or are attachable) to headpiece 24 such that the opening created by prongs 92 faces in a direction other than outwards from infant 12 (e.g., as shown in FIG. 17). For example, in FIG. 18, the opening between prongs 92 of proximal clip 88 is oriented to face toward the body of infant 12 and the opening between prongs 92 of distal clip 90 is oriented to face the front of infant 12. This alternative orientation may enhance the security of the engagement between conduit 18 or 20 and clips 88 and 90.

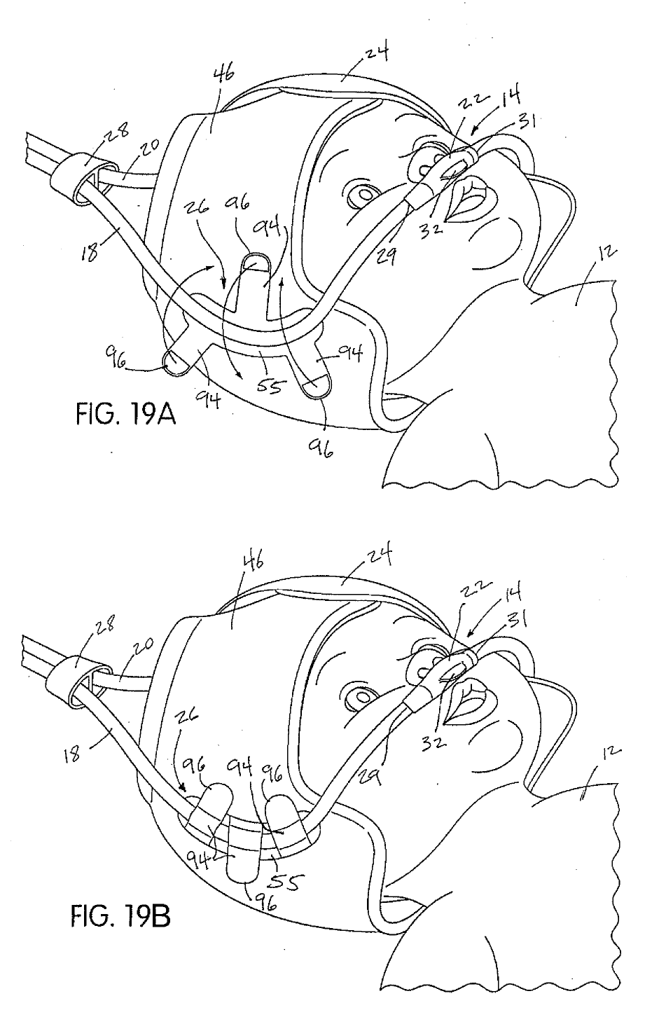

FIGS. 19A and 19B illustrate system 10, according to one embodiment of the invention, including clips 26 with an alternate configuration. As shown in FIGS. 19A and 19B, a given one of clips 26 includes one or more foldable tabs 94. Each tab 94 is permanently fixed at one end thereof to a main body 55 of clip 26, and extends from main body 55 of clip 26 to a distal end 96 of tab 94. To secure conduit 18 or 20 to clip 26, conduit 18 or 20 is laid along main body 55 of clip 26 (e.g., as shown in FIG. 19A). Then, tabs 94 are folded over the top of conduit 18 or 20 and distal ends 96 of tabs 94 are removably attached to headpiece 24. Distal ends 96 may be removably attached to headpiece 24 by hook-and-loop fasteners, adhesive, static attraction, and/or other removable attachment mechanisms. In one embodiment, rather than attaching distal ends 96 to headpiece 24, the size, shape, and/or composition of main body 55 of clip 26 may be designed to enable distal ends 96 to be removably attached to main body 55 of clip 26, rather than directly to headpiece 24. The main body 55 of clip 26 may be removably or permanently fixed to headpiece 24 in any manner described above.

It should be appreciated that the various mechanisms for securing conduits 18 and 20 in place with respect to headpiece 24 illustrated in FIGS. 1-19 are not intended to be limiting. In other embodiments, clips, channels, and/or other mechanisms that secure conduits 18 and 20 in position may be implemented without departing from the scope of the invention.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system adapted to provide a flow of gas to an airway of a patient, the system comprising:
a first conduit having a proximal end and a distal end;
a second conduit having a proximal end and a distal end;
an interface appliance having an inlet opening, an outlet opening, and one or more airway openings, the inlet opening being engaged with the proximal end of the first conduit such that the flow of gas is provided from the first conduit to the interface appliance at the engagement between the inlet opening and the proximal end of the first conduit, the one or more airway openings being adapted to deliver a portion of the flow of gas to the airway of the patient, and the outlet opening being engaged with the proximal end of the second conduit such that a portion of the flow of gas is released from the interface appliance into the second conduit at the engagement between the outlet opening and the proximal end of the second conduit; and
a retention assembly that retains the interface appliance in position with respect to the airway of the patient, wherein the retention assembly engages the first and second conduits such that if the interface appliance is in position with respect to the airway of the patient the first conduit and the second conduit run laterally across the patient's face on each side of the interface appliance underneath the eyes of the patient.

2. The system of claim 1, wherein the retention assembly engages the first and second conduits such that first and second conduits run from the engagements with the retention assembly to the top of the patient's head.

3. The system of claim 1, further comprising a gas source that communicates with the distal end of the first conduit to provide the flow of gas to the first conduit.

4. The system of claim 1, further comprising an exhaust that exhausts the portion of the flow of gas that is released from the interface appliance into the second conduit.

5. The system of claim 4, wherein the exhaust is disposed at or near the distal end of the second conduit.

6. The system of claim 1, further comprising:
a conduit couple that engages the distal end of the first conduit and the distal end of the second conduit, the conduit couple comprising:
a flow inlet that receives the flow of gas;
a flow outlet that communicates the flow of gas to the distal end of the first conduit; and
an exhaust that communicates with the second conduit to exhaust the portion of the flow of gas that is released from the interface appliance into the second conduit.

7. The system of claim 6, wherein the conduit couple further comprises a swivel that enables the flow outlet and the exhaust to swivel with respect to the flow inlet.

8. A system adapted to provide a flow of gas to an airway of a patient, the system comprising:
a first conduit having a proximal end and a distal end;
a second conduit having a proximal end and a distal end;
an interface appliance having a first opening, a second opening, and one or more airway openings, the first opening being engaged with the proximal end of the first conduit, the one or more airway openings being adapted to be in fluid communication with the airway of the patient, and the second opening being engaged with the proximal end of the second conduit; and
a retention assembly that retains the interface appliance in position with respect to the airway of the patient, wherein the retention assembly comprises clips that engage the first and second conduits between their distal and proximal ends such that near the engagements between the clips and the first and second conduits (i) the portions of the first and second conduits between the engagements and the proximal ends of the first and second conduits are oriented in directions that are roughly lateral to the patient's head and (ii) the portions of the first and second conduits between the engagements and the distal ends of the first and second conduits are oriented in directions that are roughly longitudinal to the patient's head.

9. The system of claim 8, wherein the direction of orientation of the portion of the first conduit between the engagement with one or more of the clips and the proximal end of the first conduit and the direction of orientation of the portion of the first conduit between the engagement with one or more of the clips and the distal end of the first conduit forms a first predetermined angle, and wherein the direction of orientation of the portion of the second conduit between the engagement with one or more of the clips and the proximal end of the second conduit and the direction of orientation of the portion of the second conduit between the engagement with one or more of the clips and the distal end of the second conduit forms a second predetermined angle.

10. The system of claim 9, wherein the first predetermined angle and the second predetermined angle are approximately 90 degrees.

11. The system of claim 9, wherein the first predetermined angle and the second predetermined angle are between about 60 degrees and about 120 degrees.

12. The system of claim 9, wherein the clips comprise a first clip that engages the first conduit and a second clip that engages the second conduit, and wherein the engagement of the first clip flexes the first conduit by about the first predetermined angle and the engagement of the second clip flexes the second conduit by about the second predetermined angle.

13. The system of claim 12, wherein the first clip and the second clip form arcuate paths for the first and second conduits.

14. The system of claim 8, wherein the clips comprise flanges that extend outward from the clips to releasably engage the first and second conduits.

15. The system of claim 8, further comprising a headpiece that engages the patient's head, and wherein the clips comprise one or more clips that are removably attachable to the headpiece.

16. A system adapted to provide a flow of gas to an airway of a patient the system comprising:
a first conduit having a proximal end and a distal end;
a second conduit having a proximal end and a distal end;
an interface appliance having a first opening, a second opening, and one or more airway openings, the first opening being engaged with the proximal end of the first conduit, the one or more airway openings being adapted to be in fluid communication with the airway of the patient, and the second opening being engaged with the proximal end of the second conduit; and
a retention assembly that retains the interface appliance in position with respect to the airway of the patient, wherein the retention assembly comprises clips that removably engage the first and second conduits between their distal and proximal ends.

17. The system of claim 16, wherein the clips comprise a plurality of relatively rigid flanges that protrude from the clips to engage the first and second conduits.

18. The system of claim 16, wherein the clips are formed that the first and second conduits can be drawn through the engagement between the first and second conduits and the clips.

19. The system of claim 16, further comprising a headpiece that engages the patient's head, and wherein the clips comprise one or more clips that are removably attachable to the headpiece.

20. The system of claim 19, wherein the one or more clips are removably attachable to the headpiece by virtue of hook-and-loop fasteners incorporated into the one or more clips and the headpiece.

21. A system adapted to provide a flow of gas to an airway of a patient, the system comprising:
a first conduit having a proximal end and a distal end;
a second conduit having a proximal end and a distal end;
an interface appliance having a first conduit opening, a conduit opening, and one or more airway openings, the first conduit opening being engaged with the proximal end of the first conduit, the one or more airway openings being adapted to communicate with the airway of the patient, and the outlet opening being engaged with the proximal end of the second conduit; and
a retention assembly that retains the interface appliance in position with respect to the airway of the patient, wherein the retention assembly engages the first and second conduits such that if the interface appliance is in position with respect to the airway of the patient the first conduit and the second conduit run laterally across the patient's face on each side of the interface appliance underneath the eyes of the patient.

22. The system of claim 21, wherein the retention assembly engages the first and second conduits such that first and second conduits run from the engagements with the retention assembly to the top of the patient's head.

23. The system of claim 21, further comprising a gas source that communicates with the distal end of the first conduit to provide the flow of gas to the first conduit.

24. The system of claim 21, further comprising an exhaust that exhausts the portion of the flow of gas that is released from the interface appliance into the second conduit.

25. The system of claim 24, wherein the exhaust is disposed at or near the distal end of the second conduit.

26. The system of claim 21, further comprising:
a conduit couple that engages the distal end of the first conduit and the distal end of the second conduit, the conduit couple comprising:
a flow inlet that receives the flow of gas;
a flow outlet that communicates the flow of gas to the distal end of the first conduit; and
an exhaust that communicates with the second conduit to exhaust the portion of the flow of gas that is released from the interface appliance into the second conduit.

27. The system of claim 26, wherein the conduit couple further comprises a swivel that enables the flow outlet and the exhaust to swivel with respect to the flow inlet.

28. A system adapted to provide a flow of gas to an airway of a patient the system comprising:
a first conduit having a proximal end and a distal end;
a second conduit having a proximal end and a distal end;
an interface appliance having a first opening, a second opening, and one or more airway openings, the first opening being engaged with the proximal end of the first conduit, the one or more airway openings being adapted to be in fluid communication with the airway of the patient, and the second opening being engaged with the proximal end of the second conduit; and
a retention assembly that retains the interface appliance in position with respect to the airway of the patient, wherein the retention assembly comprises a headgear that is adapted to be carried on the head of the patient and to retain the first and second conduit in position with respect to the head of the patient, the retention assembly being configured such that the position of the first and second conduits with respect to the head of the patient is adjustable by adjusting the position of the first and second conduits with respect to the headgear.

29. The system of claim 28, wherein the retention assembly further comprises a plurality of clips configured to grippingly retain the first and second conduit.

30. The system of claim 29, wherein one or more of the plurality of clips are adapted to be removably attachable to the headgear such that the position of the first and/or second conduit can be adjusted with respect to the headgear by removably attaching one or more of the plurality of clips at different locations on the headgear.

31. A flow coupling that couples a flow of gas from a gas source to one or more conduits that deliver at least a portion of the flow of gas to an airway of a patient, the flow coupling comprising:
a flow inlet that accepts the flow of gas into the flow coupling,
an outlet port that outlets at least a portion of the flow of gas to a first conduit;
a first chamber formed within the flow coupling that communicates the flow inlet with the flow outlet;
a release valve that releases gas from the first chamber to atmosphere, wherein the release valve is configured to release gas from the first chamber to atmosphere ensure that the pressure of the flow of gas delivered to the patient does not exceed a predetermined level; and
a swivel that enables the flow inlet to rotate longitudinally independent from the outlet port.

32. The flow coupling of claim 31, further comprising
an exhaust port that receives gas from a second conduit, wherein the gas received from the second conduit includes gas that is delivered by the second conduit from the airway of the patient to the exhaust port;
an exhaust opening formed in the flow coupling that releases to atmosphere the gas that has been delivered to the exhaust port by the second conduit; and
a second chamber formed within the flow coupling that communicates the exhaust port with the exhaust opening.

33. The flow coupling of claim 31 further comprising a housing having a first housing section and a second housing section, and wherein the swivel is formed at an interface between the first housing section and the second housing section.

34. The flow coupling of claim 33, wherein the first housing section provides the flow inlet, the second housing section provides the outlet port, and the first housing section and the second housing section cooperate to provide the first chamber.

35. The flow coupling of claim 34, wherein the first housing section comprises a first chamber opening defined by a first housing section rim, wherein the second housing section comprises a second chamber opening defined by a second housing section rim, and wherein the interface between the first housing section and the second housing section is formed by an interface between the first housing section rim and the second housing section rim.

36. The flow coupling of claim 35, wherein the first housing section rim fits securely within the second chamber opening formed by the second housing section rim.

37. The flow coupling of claim 35, wherein the second housing section rim fits securely within the first chamber opening formed by the first housing section rim.
